(19) United States
(12) Patent Application Publication
(10) Pub. No.: US 2016/0015607 A1
(43) Pub. Date: Jan. 21, 2016

(54) Shaving Oil Wipe

(71) Applicant: John Kevin Stevens, Akron, OH (US)

(72) Inventor: John Kevin Stevens, Akron, OH (US)

(21) Appl. No.: 14/701,637

(22) Filed: May 1, 2015

(51) Int. Cl. A61K 8/02; B65D 85/62; A61K 8/67; A61Q 9/02; A61K 8/92

(52) U.S. Cl. CPC A61K 8/0208; A61Q 9/02; A61K 8/922; A61K 8/678; B65D 85/62; A61K 2800/87

(57) ABSTRACT

A shaving oil wipe made from combining a wipe or wipe-type product with natural fats or oils for shaving. The shaving oil wipe includes a package unit and at least one shaving oil wipe received in the package unit. The shaving oil wipe includes a fibrous carrier and a shaving oil composed of oils from either natural or synthetic sources. The shaving oil is disposed on the surface of the fibrous carrier or impregnated on the fibrous carrier. When the shaving oil of the shaving oil wipe is applied on a user's skin, a layer of oil is produced on the skin to provide good lubrication for shaving. Convenience of usage, good portability, and efficient application of shaving oil to skin are advantages.

### BRIEF SUMMARY OF THE INVENTION

This invention provides a shaving oil in a wipe packaging to provide an even application of shaving oil to skin. To achieve this, a shaving oil wipe unit includes at least one wipe of fibrous absorbent material, impregnated with shaving oil, folded and enclosed in an outer package made of foil or foil-lined material to keep the shaving oil contents in place until opened by the user.

### DESCRIPTION OF THE DRAWINGS

See attached scan of invention

FIG. 1 shows the front of the sealed shaving oil wipe package.

FIG. 2 shows the back of the sealed shaving oil wipe package.

FIG. 3 shows the folded shaving oil wipe inside the foil or foil-lined package.

FIG. 4 shows the unfolded shaving oil wipe, ready to be used by the user

### DETAILED DESCRIPTION OF THE INVENTION

A folded shaving oil wipe is enclosed in a foil or foil-lined package as shown in FIG. 3. The foil or foil-lined package is then sealed around the edges to enclose the shaving oil wipe and prevent the shaving oil from leaving the package until it is opened by the user.

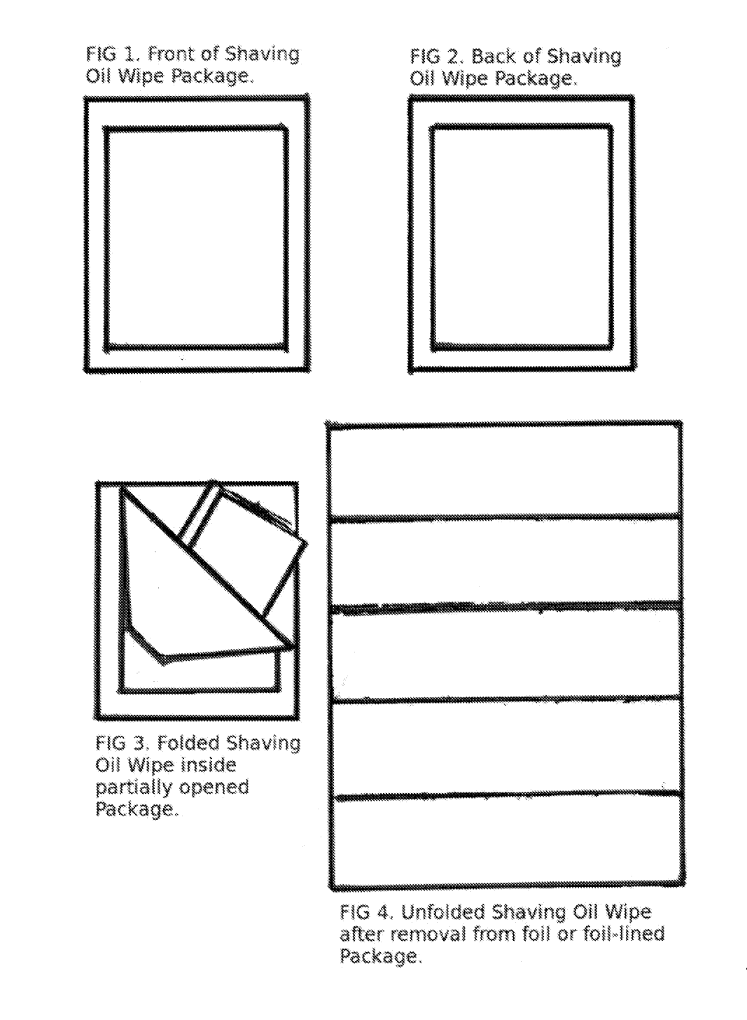

FIG. 1 and FIG. 2 show the sealed package with the shaving oil wipe folded and enclosed inside.

FIG. 4 shows the unfolded wipe once removed from the package. The user applies the shaving oil by rubbing the wipe over the skin. Shaving oil is deposited on the skin.

The shaving oil wipe unit displayed in FIG. 1 and FIG. 2 is convenient, portable, and provides efficient application of shaving oil to skin to be shaved. The invention has achieved the expected objectives.

The shaving oil is made from or derived from either individual or combinations of the following list of natural fats and oils: 1,8-cineole, alpha-linolenic acid, alpha-pinene, alpha-terpinene, alpha-terpineol, alpha-terpinolene, avocado oil, apricot oil, arachidic acid, babassu oil, behenic acid, beta-caryophyllene, borage oil, camellia oil, canola oil, caster oil, clove oil, coconut oil, corn oil, cottonseed oil, eugenol, eugenyl acetate, evening primrose oil, grapeseed oil, hydrogenated cottonseed oil, hydrogenated palm kernel oil, lignoceric acid, linoleic acid, maleated soybean oil, meadowfoam oil, myristic acid, oleic acid, olive oil, p-cymene, palm kernel oil, rapeseed oil, palmitic acid, palmitoleic acid, stearic acid, terpinen-4-ol, tocopherol, Y-terpinene, rose hip oil, sunflower oil, soybean oil, tea tree oil, and mixtures thereof, with or without the addition of other oil ingredients and fragrances.

An example shaving oil to be used in a shaving wipe is comprised of grapeseed oil, olive oil, tocopherol, clove oil, and tea tree oil. As a further example, the tea tree oil may be excluded without changing the utility of the remaining oils for their use as a skin lubricant for shaving.

Example 2 of a shaving oil to be used in a shaving oil wipe is comprised of grapeseed oil as a skin lubricant for shaving. Other oils are not necessary to be included in the shaving oil wipe for skin lubrication.

Example 3 is the use, of olive oil as a shaving oil in, a shaving oil wipe. As with Example 2, other oils are not necessary.

The above fats and oils are not emulsified, which is essential for prolonging the shelf life of the product without the addition of ingredients whose sole purpose for inclusion is to act as a preservative for the final product.

Since the invention disclosed herein may be embodied in other specific forms without departing from the spirit of general characteristics thereof, some of which forms have been indicated, the embodiments described herein are to be considered in all respects illustrative and not restrictive. The scope of the invention is to be achieved by the appended claims, rather than by the foregoing description, and all changes which come within the meaning

### PATENT CITATIONS

| Cited Patent | Filing date | Publication date | Applicant | Title |
|---|---|---|---|---|
| WO1998032416A1 | Jan. 20, 1998 | Jul. 30, 1998 | Proctor & Gamble | Method for removing |
| | | | | make-up from skin |
| WO2001021501A1 | Sep. 20, 2000 | Mar. 29, 2001 | Proctor & Gamble | Article for the delivery of |
| | | | | foam products |
| WO2001050910A1 | Jan. 9, 2001 | Jul. 19, 2001 | Burgess Agee Kirsten D | A method of |
| | | | | shaving and a |
| | | | | dispensing apparatus |
| | | | | therefor |
| EP0365160A2 | Sep. 28, 1989 | Apr. 25, 1990 | Scott Paper Company | Wet wipes |
| EP1676559A2 | Dec. 23, 2005 | Jul. 5, 2006 | L'oreal | Cosmetic or |
| | | | | dermatological article |
| | | | | containing a support |
| | | | | which is soluble in |
| | | | | water. |
| EP1171094 B1 | Apr. 20, 2000 | Jun. 28, 2006 | Kimberly-Clark Worldwide, Inc. | Wet Wipes |
| | | | | having skin health |
| | | | | benefits. |
| US20060292107 | Jun. 24, 2005 | Dec. 28, 2008 | McCormick Benjamin W | Solid shaving |
| | | | | composition and |
| | | | | packaging system |

and range of equivalency the claims are intended to be embraced therein.

I claim:

1. A shaving oil wipe unit comprising:
one shaving oil wipe with shaving oil disposed on the surface of the shaving oil wipe or impregnated in the shaving oil wipe with a lubricating layer produced on the surface of the skin when the shaving oil wipe is applied to the skin and a package unit having an internal accommodation chamber therein; and a shaving oil wipe received in the accommodation chamber in foldable or rolled form.

2. A shaving oil wipe unit comprising:
more than one shaving oil wipe with shaving oil disposed on the surface of the shaving oil wipe or impregnated in the shaving oil wipe with a lubricating layer produced on the surface of the skin when the shaving oil wipe is applied to the skin and a package unit having an internal accommodation chamber therein; and more than one shaving oil wipe received in the accommodation chamber in foldable or rolled form.

3. The shaving oil wipe unit according to claim 1, wherein the shaving oil is made from or derived from either an individual or combination of natural fats and oils, wherein the natural fats and oils are 1,8-cineole, alpha-linolenic acid, alpha-pinene, alpha-terpinene, alpha-terpineol, alpha-terpinolene, avocado oil, apricot oil, arachidic acid, babassu oil, behenic acid, beta-caryophyllene, borage oil, camellia oil, canola oil, caster oil, clove oil, coconut oil, corn oil, cottonseed oil, eugenol, eugenyl acetate, evening primrose oil, grapeseed oil, hydrogenated cottonseed oil, hydrogenated palm kernel oil, lignoceric acid, linoleic acid, maleated soybean oil, meadowfoam oil, myristic acid, oleic acid, olive oil, p-cymene, palm kernel oil, rapeseed oil, palmitic acid, palmitoleic acid, stearic acid, terpinen-4-ol, tocopherol, Y-terpinene, rose hip oil, sunflower oil, soybean oil, tea tree oil, and mixtures thereof, with or without the addition of other oil ingredients and fragrances.

4. The shaving oil wipe unit according to claim 1, wherein the shaving oil is made from or derived from natural fat and oils, wherein the natural fats and oils are grapeseed oil, clove oil, olive oil, tocopherol, tea tree oil, either individually and mixtures thereof, with or without the addition of other oil ingredients and fragrances.

5. The shaving oil wipe unit according to claim 1, wherein the shaving oil wipe is composed of a single type of fibrous absorbent fiber or a combination of two or more different types of fibrous absorbent fibers with shaving oil according to claim 3 or according to claim 4, disposed on the surface of the fibrous absorbent fibers or impregnated in the fibrous absorbent fibers with a lubricating layer produced on the skin when the fibrous absorbent materials are applied to the skin.

6. The shaving oil wipe unit according to claim 1, wherein the shaving oil wipe is composed of a combination of one or more fibrous absorbent fibers that absorb the shaving oil according to claim 3 or according to claim 4, and one or more fibers that do not absorb the shaving oil with shaving oil disposed on the surface of the fibrous absorbent fibers or impregnated in the fibrous absorbent fibers with a lubricating layer produced on the skin when the fibrous absorbent materials are applied to the skin.

7. The shaving oil wipe unit according to claim 1, wherein the package unit is foil or foil-lined when used in combination with the shaving oil wipe unit according to claim 3.

8. The shaving oil wipe unit according to claim 1, wherein the package unit is foil or foil-lined when used in combination with the shaving oil wipe unit according to claim 4.
